(19) United States
(12) Patent Application Publication
(10) Pub. No.: US 2022/0258075 A1
(43) Pub. Date: Aug. 18, 2022

(54) FILTER ASSEMBLY FOR HIGH VOLUME EVACUATION

(71) Applicant: SAINT-GOBAIN PERFORMANCE PLASTICS CORPORATION, Solon, OH (US)

(72) Inventors: Boon Pheng TANG, Galena, OH (US); Vietlinh Nguyen, Cleveland, OH (US); Kenneth Renfrew, Huntersville, NC (US); Cristian Rios Castro, Springfield, VA (US); Gary Golovan, Pepper Pike, OH (US); Bruce Golovan, Pepper Pike, OH (US)

(21) Appl. No.: 17/651,372

(22) Filed: Feb. 16, 2022

Related U.S. Application Data
(60) Provisional application No. 63/149,878, filed on Feb. 16, 2021.

(51) Int. Cl. B01D 29/085; B01D 35/02; A61C 17/08

(52) U.S. Cl. CPC B01D 29/085; B01D 2201/204; A61C 17/08; B01D 35/02

(57) ABSTRACT

Systems and methods are disclosed that include providing a filter assembly having a filter housing and a filter medium disposed within and/or carried by the filter housing. The filter medium is configured to be exposed to an external environment and collect, filter, or otherwise remove aerosols (which may be contaminated with bacteria, viruses, or a combination thereof) from the external environment when coupled to an evacuation component, such as a dental high vacuum evacuation (HVE) line.

### CROSS-REFERENCE TO RELATED APPLICATION(S)

This application claims priority under 35 U.S.C. § 119(e) to U.S. Provisional Application No. 63/149,878, entitled “FILTER ASSEMBLY FOR HIGH VOLUME EVACUATION,” by Boon Pheng TANG et al., filed Feb. 16, 2021, which is assigned to the current assignee hereof and incorporated herein by reference in its entirety.

### BACKGROUND OF THE INVENTION

Filter assemblies are frequently used in the medical field to ensure a sterile environment and/or safe operating conditions. Some filter assemblies utilize a filter medium that may filter out or separate bacterium, particulates, viruses or other contaminants from a fluid or gas passing through the filter assembly. Some traditional filters may not prevent cross-contamination or effectively separate bacterium, particulates, or viruses from the surrounding environment to prevent cross-contamination. Because effective and reliable operation of such filter assemblies remains critical to sterile medical procedures, the medical industry continues to demand improved filters for various applications.

### BRIEF DESCRIPTION OF THE DRAWINGS

So that the manner in which the features and advantages of the embodiments are attained and can be understood in more detail, a more particular description may be had by reference to the embodiments thereof that are illustrated in the appended drawings.

FIG. 1 is an oblique view of a filter assembly according to an embodiment of the disclosure.

FIG. 2 is a cross sectional view of a filter assembly according to an embodiment of the disclosure.

FIG. 3 is an oblique exploded view of a filter assembly and a dental high vacuum evacuation (HVE) component according to an embodiment of the disclosure.

FIG. 4 is an oblique view of a filter assembly having a cap according to an embodiment of the disclosure.

FIG. 5 is an orthogonal side view of a filter assembly having a cap according to an embodiment of the disclosure.

FIG. 6 is a flowchart of a method of filtering aerosols from an external environment according to an embodiment of the disclosure.

FIG. 7 is a flowchart of a method of forming a filter assembly according to an embodiment of the disclosure.

The use of the same reference symbols in different drawings indicates similar or identical items.

### DETAILED DESCRIPTION

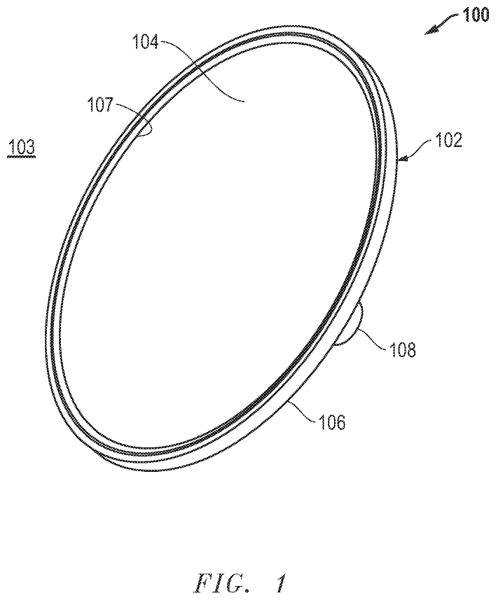

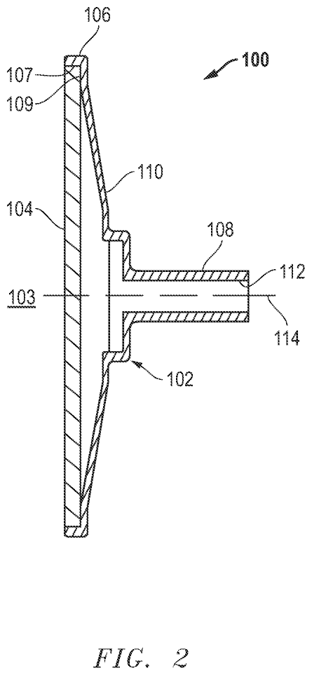

FIGS. 1 and 2 show an oblique view and a cross sectional view of a filter assembly 100 according to an embodiment of the disclosure. The filter assembly 100 may generally comprise a filter housing 102 and a filter medium 104 carried by the filter housing 102 and at least partially directly exposed to an external environment 103 to be filtered by the filter assembly 100. The filter housing 102 may generally comprise an inlet portion 106 and an outlet portion 108. In some embodiments, the inlet portion 106 and/or the outlet portion 108 may be circular and/or annularly disposed about a central axis 114. However, in some embodiments, the inlet portion 106 and/or the outlet portion 108 may be elliptical, rectangular, square, regularly or irregularly shaped, or comprise any other shape, profile, or combination of shapes and/or profiles. In some embodiments, the inlet portion 106 may be axially aligned with the outlet portion 108 along the central axis 114. Additionally, in some embodiments, the filter housing 102 may comprise a connecting portion 110 disposed between the inlet portion 106 and the outlet portion 108. In some embodiments, the connecting portion 110 may be at least partially tapered between the inlet portion 106 and the outlet portion 108.

The inlet portion 106 may generally comprise a larger major dimension than the outlet portion 108. The major dimension may generally be defined as the largest measurement of the inlet portion 106 and the outlet portion 108. For example, the major dimension may be a diameter of a circular-shaped inlet portion 106 and/or the outlet portion 108, a longest side of a rectangular-shaped inlet portion 106 and/or the outlet portion 108, or either side of a square-shaped inlet portion 106 and/or the outlet portion 108. Thus, in some embodiments, the major dimension may be a diameter of each of the inlet portion and the outlet portion. In some embodiments, the major dimension of the inlet portion may be at least 25 millimeters (mm), at least 50 mm, at least 75 mm, at least 100 mm, at least 125 mm, at least 150 mm, at least 175 mm, at least 200 mm, at least 250 mm, at least 300 mm, at least 350 mm, or even larger such as at least 600 mm or at least 1200 mm. It will be appreciated that the major dimension of the inlet portion 106 may be selected based on an intended application for the filter assembly. In some embodiments, the major dimension of the inlet portion 106 and major dimension of the outlet portion 108 may comprise a relative size ratio. In some exemplary embodiments, the ratio of the major dimension of the inlet portion 106 to the major dimension of the outlet portion 108 may be between 5.0:1 to 6.8:1 for a filter assembly 100 comprising an inlet portion 106 with a major dimension of about 75 mm. In other exemplary embodiments, the ratio of the major dimension of the inlet portion 106 to the major dimension of the outlet portion 108 may be between 10:1 to 13.6:1 for a filter assembly 100 comprising an inlet portion 106 with a major dimension of about 150 mm.

In some embodiments, the major dimension of the outlet portion 108 may be at least 10 millimeters (mm), at least 11 mm, at least 12 mm, at least 13 mm, at least 14 mm, at least 15 mm, at least 20 mm, at least 25 mm, or even greater such as at least 50 mm, at least 100 mm, at least 300 mm, or at least 600 mm. In some embodiments, the major dimension of the outlet portion 108 may be not greater than 300 mm, not greater than 150 mm, not greater than 50 mm, not greater than 25 mm, not greater than 20 mm, not greater than 19 mm, not greater than 18 mm, not greater than 17 mm, not greater than 16 mm, or not greater than 15 mm. In particular embodiments, the major dimension of the outlet portion may be about 11 mm. In other particular embodiments, the major dimension of the outlet portion 108 may be about 15 mm. Further, it will be appreciated that the major dimension of the outlet portion 108 may be between any of these minimum and maximum values, such as at least 10 mm to not greater than 25 mm, or even at least 11 mm to not greater than 15 mm. It will be appreciated that the major dimension of the outlet portion 108 may be selected to fit a particular intended application.

In some embodiments, the filter housing 102 may be formed by additive manufacturing, blow molding, injection molding, machining, thermoforming, or any combination thereof. In some embodiments, the filter housing 102 may be formed from a polymeric material. In some embodiments, the filter housing 102 may be formed from a recyclable polymeric material. For example, in some embodiments, the filter housing 102 may be formed from PTFE.

The inlet portion 106 of the filter assembly 100 may comprise a lip 107 that forms a cavity or an opening in the inlet portion 106. The filter medium 104 may be disposed within the opening in the inlet portion 106 of the filter housing 102. Accordingly, it will be appreciated that the filter medium 104 may comprise a complementary shape to the inlet portion 106 of the filter housing (e.g., circular disc shape). In some embodiments, the filter medium 104 may be disposed within the opening, such that the filter medium 104 is in contact with the lip 107 of the inlet portion 106 and/or a back surface 109 of the inlet portion 106. In some embodiments, the filter medium 104 may also be in contact with other portions (e.g., connecting portion 110) of the filter housing 102. In some embodiments, the filter medium 104 may comprise a uniform thickness. However, in other embodiments, the filter medium 104 may comprise a non-uniform thickness.

In some embodiments, the filter medium 104 may be carried by the filter housing 102. In some embodiments, the filter medium 104 may be secured to the filter housing 102. In some embodiments, the filter medium 104 may be secured to the filter housing 102 through an interference fit between the filter medium 102 and the opening in the inlet portion 106 of the filter housing 102, through an adhesive applied between the filter medium 104 and the filter housing 102, through ultrasonic welding the filter medium 104 to the filter housing 102, or any combination thereof. In some embodiments, the filter medium 104 may be secured to the filter housing 102 via any of these means and be secured to lip 107 of inlet portion 106 of the filter housing 102, secured to back surface 109 of inlet portion 106 of the filter housing 102, or a combination thereof. In other embodiments, the filter medium 104 may be secured to other portions (e.g., connecting portion 110) of the filter housing 102.

The filter medium 104 may also be at least partially directly exposed to the external environment 103 to be filtered by the filter assembly 100. In some embodiments, the at least 75%, at least 80%, at least 85%, at least 90%, at least 95%, at least 96%, at least 97%, at least 98%, at least 99%, or 100% of an outer surface of the filter medium 104 may be exposed to the external environment 103. In some embodiments, the filter medium 104 may comprise a single filter medium element. However, in other embodiments, the filter medium 104 may comprise multiple filter medium elements, which may be layered and/or which may be joined to a form a unitary filter medium 104 comprising multiple layers. In alternative embodiments, the filter medium 104 may comprise a filter medium housing that carries the filter medium 104 and which is configured to be selectively coupled to the filter housing 102.

In operation, the filter assembly 100 may be connected to an evacuation component, such as a dental high vacuum evacuation (HVE) line, an optometry or ophthalmology suction line, a surgical fluid evacuation line used in various medical procedures, a veterinarian suction line, or the like. Additionally, in some embodiments, the filter assembly 100 may be connected to evacuation equipment such as that used in commercial food service operations. When connected to an evacuation component, the filter assembly 100 may be configured to remove aerosols from the external environment 103. The filter housing 102 may comprise a flow passage 112 connecting the inlet portion 106 and the outlet portion 108 to facilitate removal of the aerosols from the external environment 103. Accordingly, the aerosols removed from the external environment 103 may pass through the filter medium 104, through the flow passage 112, and into the evacuation component coupled to the filter assembly 100.

In some embodiments, the filter medium 104 may be configured to remove bacteria, viruses, other contaminants or chemicals (e.g., blood, mercury, etc.) from the external environment 103. Accordingly, in some embodiments, the filter medium 104 may comprise an antibacterial agent, an antimicrobial agent, an antiviral agent, or a combination thereof. Further, the filter medium 104 and/or filter assembly 100 may be configured to prevent the backflow of bacteria, viruses, other contaminants or chemicals (e.g., blood, mercury, etc.) through the filter medium 104 into the external environment 103. In some embodiments, the filter medium 104 may comprise a HEPA (“high-efficiency particulate air) or Ultra HEPA filter medium. In some embodiments, the filter medium 104 may comprise a HEPA grade of at least H10 that captures at least 85% of all particles 0.3 micron or bigger. In some embodiments, the filter medium 104 may comprise a HEPA grade of at least H11 that captures at least 95% of all particles 0.3 micron or bigger. In some embodiments, the filter medium 104 may comprise a HEPA grade of at least H12 that captures at least 99.5% of all particles 0.3 micron or bigger. In some embodiments, the filter medium 104 may comprise a HEPA grade of at least H13 that captures at least 99.95% of all particles 0.3 micron or bigger. In some embodiments, the filter medium 104 may comprise a HEPA grade of at least H14 that captures at least 99.995% of all particles 0.3 micron or bigger.

Further, in some embodiments, the filter housing 102 may comprise or be treated with an antibacterial agent, an antimicrobial agent, an antiviral agent, or a combination thereof, which may provide the filter housing 102 with antibacterial, antimicrobial, and/or antiviral properties. In some embodiments, the filter assembly 100 may reduce the potential for cross-patient contamination which can arise from traditional evacuation components not being replaced or cleaned between procedures.

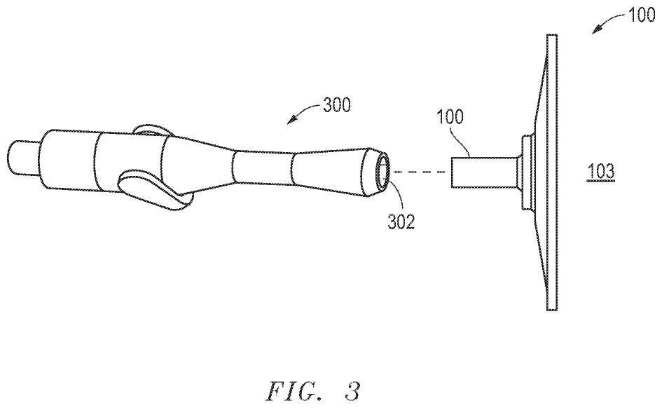

FIG. 3 shows an oblique exploded view of a filter assembly 100 and a dental high vacuum evacuation (HVE) component 300 according to an embodiment of the disclosure. As stated, the filter assembly 100 may generally be configured to be coupled to an evacuation component, such as the dental HVE component 300 shown. More specifically, the outlet portion 108 of the filter housing 102 may be configured for insertion into an orifice 302 of the evacuation component 300. In some embodiments, the outlet portion 108 of the filter housing 102 may be configured to press fit into the orifice 302 of the evacuation component 300. Accordingly, the outlet portion 108 of the filter housing 102 may be free of a coupling or fitting. This type of tight tolerance clearance fit or interference fit may ensure the filter assembly 100 remains connected to the evacuation component 300 during operation. However, in alternative embodiments, the outlet portion 108 of the filter housing 102 may comprise a coupling or fitting based on the application for connection to a complementary coupling or fitting on the end of the evacuation component 300.

The filter assembly 100 may be configured for operation between 0.034 kPa (0.005 psi) and 1723 kPa (250 psi). This pressure may generally be generated by the evacuation equipment that the evacuation component 300 is coupled to. The filter assembly 100 may be configured for operation at a flow rate of at least 0.005 cubic meters per second (m³/sec), at least 0.010 m³/sec, at least 0.015 m³/sec, at least 0.025 m³/sec, at least 0.05 m³/sec, at least 0.075 m³/sec, at least 0.10 m³/sec, at least 0.25 m³/sec, at least 0.50 m³/sec, at least 0.75 m³/sec, at least 1.0 m³/sec, at least 5.0 m³/sec, at least 10.0 m³/sec, or at least 25.0 m³/sec through the filter assembly 100. The filter assembly 100 may generally be configured to remove aerosols from the external environment 103 when the filter assembly 100 is placed in proximity to a surgical location of a medical procedure. As used herein, medical procedure refers to medical procedures, dental procedures (including but not limited to dental cleanings), surgical procedures, or veterinary procedures. The filter assembly 100 may be configured to remove aerosols from the external environment 103 when the filter assembly 100 (e.g., filter medium 104) is placed at a distance of between 0 mm and 350 mm from the surgical location of the medical procedure. The filter assembly 100 may also be configured for filtering aerosols in various industries. In some embodiments, the surgical location may be the mouth of a subject for a dental operation. In some embodiments, the surgical location may be an incision on a subject for a medical operation. Additionally, it will be appreciated that the subject may comprise a human or an animal which may expel aerosols that require removal to ensure the safety of the subject or other personnel involved in the medical procedure. However, the filter assembly 100 may be used in any industry or application (e.g., food service) requiring the removal of aerosols from an external environment for safety.

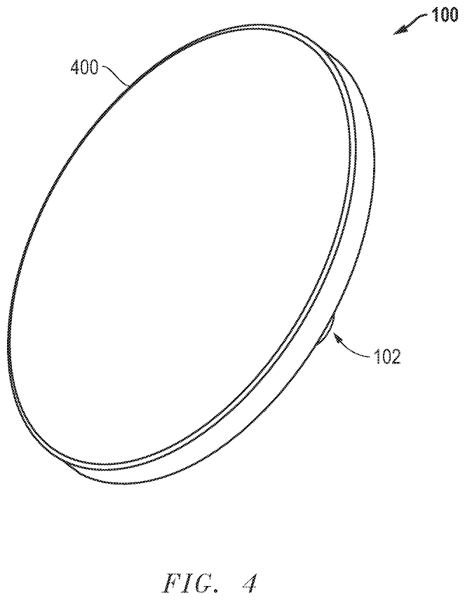

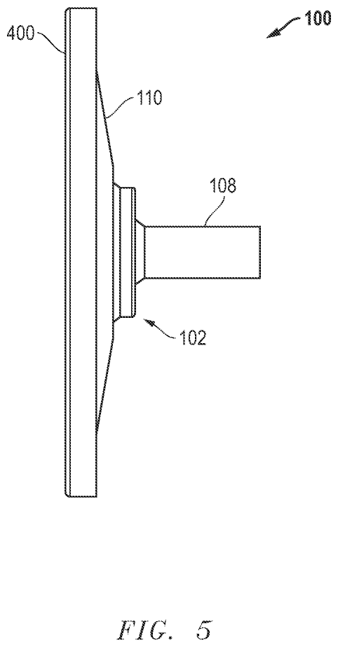

FIGS. 4 and 5 show an oblique view and an orthogonal view of a filter assembly 100 having a cap 400 according to an embodiment of the disclosure. The filter assembly 100 may generally be configured for a single use for a medical procedure. This may prevent cross-contamination between various subjects undergoing medical procedures in a common location. The filter assembly 100 may be removable from the evacuation component 300 in order to be disposed. Thus, it will be appreciated that the filter assembly 100 may be disposable. In some embodiments, the filter assembly 100 may be configured to receive a cap 400 over the inlet portion 106 of the filter housing 102 that covers the filter medium 104 for disposal. In some embodiments, the filter assembly 100 may also be configured to receive a cap over or plug into the flow passage of the outlet portion 108 for disposal. In some embodiments, the cap 400 may snap onto the filter housing 102. In other embodiments, the cap 400 may screw or twist onto the filter housing 102. As such, the cap 400 may thereby effectively reduce the likelihood or altogether prevent cross-contamination from substances filtered by the filter medium 104. Additionally, in some embodiments, the cap 400 may comprise or be treated with an antibacterial agent, an antimicrobial agent, an antiviral agent, or a combination thereof, which may provide the cap 400 with antibacterial, antimicrobial, and/or antiviral properties.

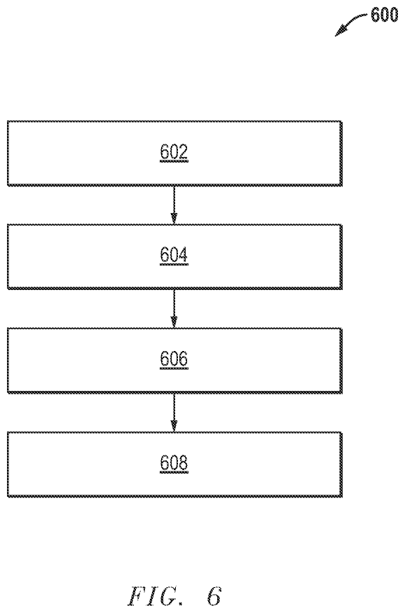

FIG. 6 is a flowchart of a method 600 of filtering aerosols from an external environment 103 according to an embodiment of the disclosure. The method 600 may begin at block 602 by providing a filter assembly 100 comprising a filter housing 102 and a filter medium 104 carried by the filter housing 102 and at least partially directly exposed to an external environment 103 to be filtered by the filter assembly 100. The method 600 may continue at block 604 by coupling the filter assembly 100 to an evacuation component 300 of a piece of evacuation equipment. In some embodiments, the evacuation component 300 may be a dental high vacuum evacuation (HVE) line. The method 600 may continue at block 606 by placing the filter assembly 100 in proximity to a surgical location of a medical procedure. The method 600 may continue at block 608 by operating the evacuation equipment to draw aerosols from the external environment 103 through the filter medium 104 and through the evacuation component 300 to remove the aerosols from the external environment 103. In some embodiments, the filter assembly 100 may be configured to remove aerosols from the external environment when the filter assembly 100 is placed at a distance of between 0 mm and 350 mm from the surgical location of the medical procedure. In some embodiments, the method 600 may also comprise moving the filter assembly 100 from the evacuation component 300 and disposing the filter assembly 100. Further in some embodiments, the method 600 may also comprise placing a cap 400 over an inlet portion 106 of the filter assembly 100 to cover the filter medium 104 for disposal, placing a cap over an outlet portion 108 of the filter assembly for disposal, or a combination thereof.

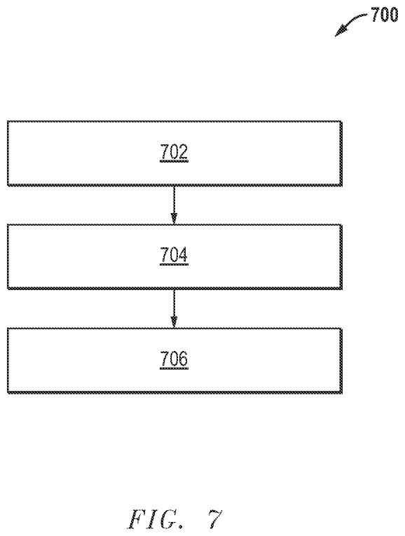

FIG. 7 is a flowchart of a method 700 of forming a filter assembly 100 according to an embodiment of the disclosure. The method 700 may begin at block 702 by forming a filter housing 102. In some embodiments, the filter housing 102 may be formed through additive manufacturing, blow molding, injection molding, machining, thermoforming, or any combination thereof. The method 700 may continue at block 704 by forming a filter medium 104. In some embodiments, forming the filter medium 104 may comprise treating the filter housing 102, the filter medium 104, or a combination thereof with an antibacterial agent, an antibacterial agent, an antiviral agent, or a combination thereof. The method 700 may continue at block 706 by coupling the filter medium 104 to the filter housing 102. In some embodiments, the filter medium 104 may be coupled to the filter housing 102 through ultrasonic welding.

In some embodiments, a filter assembly 100, method 600 of filtering aerosols, and/or a method 700 of forming a filter assembly 100 may include one or more of the following embodiments:

Embodiment 1. A filter assembly, comprising: a filter housing; and a filter medium carried by the filter housing and at least partially directly exposed to an external environment to be filtered by the filter assembly.

Embodiment 2. The filter assembly of embodiment 1, wherein the filter housing comprises an inlet portion and an outlet portion.

Embodiment 3. The filter assembly of embodiment 2, wherein the inlet portion is circular, and wherein the outlet portion is circular.

Embodiment 4. The filter assembly of embodiment 3, wherein the inlet portion is axially aligned with the outlet portion.

Embodiment 5. The filter assembly of embodiment 4, wherein the filter housing comprises a connecting portion between the inlet portion and the outlet portion.

Embodiment 6. The filter assembly of embodiment 5, wherein the connecting portion is at least partially tapered between the inlet portion and the outlet portion.

Embodiment 7. The filter assembly of any of embodiments 2 to 6, wherein the inlet portion comprises a larger major dimension than the outlet portion.

Embodiment 8. The filter assembly of embodiment 7, wherein the major dimension of the inlet portion is at least 25 mm, at least 50 mm, at least 75 mm, at least 100 mm, at least 125 mm, at least 150 mm, at least 175 mm, at least 200 mm, at least 250 mm, at least 300 mm, at least 350 mm, or larger such as at least 600 mm or at least 1200 mm.

Embodiment 9. The filter assembly of any of embodiments 1 to 8, wherein the filter assembly is configured to be coupled to an evacuation component.

Embodiment 10. The filter assembly of embodiment 9, wherein the outlet portion is configured for insertion into the evacuation component.

Embodiment 11. The filter assembly of embodiment 10, wherein the outlet portion is configured to press fit into the evacuation component.

Embodiment 12. The filter assembly of embodiment 11, wherein the outlet portion is free of a coupling or fitting.

Embodiment 13. The filter assembly of embodiment 12, wherein the evacuation component comprises a dental high vacuum evacuation (HVE) line.

Embodiment 14. The filter assembly of embodiment 9, wherein the outlet portion comprises a coupling or fitting.

Embodiment 15. The filter assembly of any of embodiments 7 to 14, wherein the major dimension of the outlet portion is at least 10 mm, at least 11 mm, at least 12 mm, at least 13 mm, at least 14 mm, at least 15 mm, or even greater such as at least 50 mm, at least 100 mm, at least 300 mm, or at least 600 mm.

Embodiment 16. The filter assembly of embodiment 15, wherein the major dimension of the outlet portion is not greater than 300 mm, not greater than 150 mm, not greater than 50 mm, not greater than 25 mm, not greater than 20 mm, not greater than 19 mm, not greater than 18 mm, not greater than 17 mm, not greater than 16 mm, or not greater than 15 mm.

Embodiment 17. The filter assembly of any of embodiments 15 to 16, wherein the major dimension of the outlet portion is about 11 mm.

Embodiment 18. The filter assembly of any of embodiments 15 to 16, wherein the major dimension of the outlet portion is about 15 mm.

Embodiment 19. The filter assembly of any of embodiments 7 to 18, wherein a ratio of the major dimension of the inlet portion to the major dimension of the outlet portion is between 5.0:1 to 6.8:1 for a filter assembly comprising an inlet portion with a major dimension of about 75 mm.

Embodiment 20. The filter assembly of any of embodiments 7 to 18, wherein a ratio of the major dimension of the inlet portion to the major dimension of the outlet portion is between 10:1 to 13.6:1 for a filter assembly comprising an inlet portion with a major dimension of about 150 mm.

Embodiment 21. The filter assembly of any of embodiments 1 to 20, wherein the major dimension is a diameter of each of the inlet portion and the outlet portion.

Embodiment 22. The filter assembly of any of embodiments 1 to 21, wherein the filter housing is formed by additive manufacturing, blow molding, injection molding, machining, thermoforming, or any combination thereof.

Embodiment 23. The filter assembly of any of embodiments 1 to 22, wherein the filter housing is formed from PTFE.

Embodiment 24. The filter assembly of any of embodiments 1 to 23, wherein the inlet portion comprises a lip that forms an opening in the inlet portion.

Embodiment 25. The filter assembly of embodiment 24, wherein the filter medium is disposed within the opening in the inlet portion.

Embodiment 26. The filter assembly of embodiment 25, wherein the filter medium is secured to the filter housing.

Embodiment 27. The filter assembly of embodiment 26, wherein the filter medium is secured to the filter housing through an interference fit between the filter medium and the opening, through an adhesive, through ultrasonic welding, or any combination thereof.

Embodiment 28. The filter assembly of any of embodiments 1 to 27, wherein at least 75%, at least 80%, at least 85%, at least 90%, at least 95%, at least 96%, at least 97%, at least 98%, at least 99%, or 100% of an outer surface of the filter medium is exposed to the external environment.

Embodiment 29. The filter assembly of any of embodiments 1 to 23, wherein the filter medium comprises a filter medium housing.

Embodiment 30. The filter assembly of embodiment 29, wherein the filter medium housing is configured to be selectively coupled to the filter housing.

Embodiment 31. The filter assembly of any of embodiments 1 to 30, wherein the filter assembly is configured to remove aerosols from the external environment.

Embodiment 32. The filter assembly of embodiment 31, wherein the filter housing comprises a flow passage connecting the inlet portion and the outlet portion to facilitate removal of the aerosols from the external environment.

Embodiment 33. The filter assembly of embodiment 32, wherein the aerosols removed from the external environment pass through the filter medium, through the flow passage, and into an evacuation component coupled to the filter assembly.

Embodiment 34. The filter assembly of any of embodiments 1 to 33, wherein the filter medium removes bacteria, viruses, other contaminants or chemicals (e.g., blood, mercury, etc.) from the external environment.

Embodiment 35. The filter assembly of embodiment 34, wherein the filter medium comprises an antibacterial agent, an antimicrobial agent, an antiviral agent, or a combination thereof.

Embodiment 36. The filter assembly of any of embodiments 34 to 35, wherein the filter housing comprises or is treated with an antibacterial agent, an antimicrobial agent, an antiviral agent, or a combination thereof.

Embodiment 37. The filter assembly of embodiment 1, wherein the filter medium prevents backflow of bacteria, viruses, other contaminants or chemicals (e.g., blood, mercury, etc.) through the filter medium into the external environment.

Embodiment 38. The filter assembly of any of embodiments 1 to 37, wherein the filter assembly reduces the potential for cross-patient contamination which can arise from traditional evacuation components not being replaced or cleaned between procedures.

Embodiment 39. The filter assembly of any of embodiments 1 to 38, wherein the filter assembly is configured for operation between 0.005 psi (0.034 kPa) and 250 psi (1723 kPa).

Embodiment 40. The filter assembly of any of embodiments 1 to 39, wherein the filter assembly is configured for operation at a flow rate of at least 0.005 cubic meters per second (m³/sec), at least 0.010 m³/sec, at least 0.015 m³/sec, at least 0.025 m³/sec, at least 0.05 m³/sec, at least 0.075 m³/sec, at least 0.10 m³/sec, at least 0.25 m³/sec, at least 0.50 m³/sec, at least 0.75 m³/sec, at least 1.0 m³/sec, at least 5.0 m³/sec, at least 10.0 m³/sec, or at least 25.0 m³/sec through the filter assembly.

Embodiment 41. The filter assembly of any of embodiments 1 to 40, wherein the filter assembly is configured to remove aerosols from the external environment when the filter assembly is placed in proximity to a surgical location of a medical procedure.

Embodiment 42. The filter assembly of embodiment 41, wherein the filter assembly is configured to remove aerosols from the external environment when the filter assembly is placed at a distance of between 0 mm and 350 mm from the surgical location of the medical procedure.

Embodiment 43. The filter assembly of any of embodiments 41 to 42, wherein the surgical location is the mouth of a subject for a dental operation.

Embodiment 44. The filter assembly of any of embodiments 41 to 42, wherein the surgical location is an incision on a subject for a medical operation.

Embodiment 45. The filter assembly of any of embodiments 1 to 44, wherein the filter assembly is configured for a single use for a medical procedure.

Embodiment 46. The filter assembly of embodiment 45, wherein the filter assembly is removable from the evacuation component.

Embodiment 47. The filter assembly of any of embodiments 45 to 46, wherein the filter assembly is disposable.

Embodiment 48. The filter assembly of any of embodiments 45 to 47, wherein the filter assembly is configured to receive a cap over the inlet portion that covers the filter medium for disposal, over the outlet portion, or a combination thereof.

Embodiment 49. The filter assembly of embodiment 48, wherein the cap snaps onto the filter housing.

Embodiment 50. The filter assembly of embodiment 48, wherein the cap screws or twists onto the filter housing.

Embodiment 51. The filter assembly of any of embodiments 48 to 50, wherein the cap comprises or is treated with an antibacterial agent, an antimicrobial agent, an antiviral agent, or a combination thereof.

Embodiment 52. A method of filtering aerosols from an external environment, comprising: providing a filter assembly comprising a filter housing and a filter medium carried by the filter housing and at least partially directly exposed to an external environment to be filtered by the filter assembly; coupling the filter assembly to an evacuation component of a piece of evacuation equipment; placing the filter assembly in proximity to a surgical location of a medical procedure; and operating the evacuation equipment to draw aerosols from the external environment through the filter medium and through the evacuation component to remove the aerosols from the external environment.

Embodiment 53. The method of embodiment 52, wherein the evacuation component comprises a dental high vacuum evacuation (HVE) line.

Embodiment 54. The method of any of embodiments 52 to 53, wherein the filter assembly is configured to remove aerosols from the external environment when the filter assembly is placed at a distance of between 0 mm and 350 mm from the surgical location of the medical procedure.

Embodiment 55. The method of any of embodiments 52 to 54, further comprising:
removing the filter assembly from the evacuation component and disposing the filter assembly.

Embodiment 56. The method of embodiment 55, further comprising: placing a cap over an inlet portion of the filter assembly to cover the filter medium for disposal, placing a cap over an outlet portion of the filter assembly for disposal, or a combination thereof.

Embodiment 57. A method of forming a filter assembly, comprising: forming a filter housing; forming a filter medium; and coupling the filter medium to the filter housing.

Embodiment 58. The method of embodiment 57, wherein the filter housing is formed through additive manufacturing, blow molding, injection molding, machining, thermoforming, or any combination thereof.

Embodiment 59. The method of any of embodiments 57 to 58, wherein forming the filter medium comprises treating the filter housing, the filter medium, or a combination thereof with an antibacterial agent, an antibacterial agent, an antiviral agent, or a combination thereof.

Embodiment 60. The method of any of embodiments 57 to 59, wherein the filter medium is coupled to the filter housing through ultrasonic welding.

This written description uses examples to disclose the embodiments, including the best mode, and also to enable those of ordinary skill in the art to make and use the invention. The patentable scope is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

Note that not all of the activities described above in the general description or the examples are required, that a portion of a specific activity may not be required, and that one or more further activities may be performed in addition to those described. Still further, the order in which activities are listed are not necessarily the order in which they are performed.

In the foregoing specification, the concepts have been described with reference to specific embodiments. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of invention.

As used herein, the terms “comprises,” “comprising,” “includes,” “including,” “has,” “having” or any other variation thereof, are intended to cover a non-exclusive inclusion. For example, a process, method, article, or apparatus that comprises a list of features is not necessarily limited only to those features but may include other features not expressly listed or inherent to such process, method, article, or apparatus. Further, unless expressly stated to the contrary, “or” refers to an inclusive-or and not to an exclusive-or. For example, a condition A or B is satisfied by any one of the following: A is true (or present) and B is false (or not present), A is false (or not present) and B is true (or present), and both A and B are true (or present).

Also, the use of “a” or “an” are employed to describe elements and components described herein. This is done merely for convenience and to give a general sense of the scope of the invention. This description should be read to include one or at least one and the singular also includes the plural unless it is obvious that it is meant otherwise.

Benefits, other advantages, and solutions to problems have been described above with regard to specific embodiments. However, the benefits, advantages, solutions to problems, and any feature(s) that may cause any benefit, advantage, or solution to occur or become more pronounced are not to be construed as a critical, required, or essential feature of any or all the claims.

After reading the specification, skilled artisans will appreciate that certain features are, for clarity, described herein in the context of separate embodiments, may also be provided in combination in a single embodiment. Conversely, various features that are, for brevity, described in the context of a single embodiment, may also be provided separately or in any subcombination. Further, references to values stated in ranges include each and every value within that range.

What is claimed is:

1. A filter assembly, comprising:
a filter housing; and
a filter medium carried by the filter housing and at least partially directly exposed to an external environment to be filtered by the filter assembly.

2. A filter assembly, comprising:
a filter housing; and
a filter medium carried by the filter housing and at least partially directly exposed to an external environment to be filtered by the filter assembly, wherein the filter medium removes bacteria, viruses, other contaminants or chemicals from the external environment.

3. A filter assembly, comprising:
a filter housing; and
a filter medium carried by the filter housing and at least partially directly exposed to an external environment to be filtered by the filter assembly, wherein the filter assembly is configured to remove aerosols from the external environment when the filter assembly is placed in proximity to a surgical location of a medical procedure.

4. The filter assembly of claim 1, wherein the filter housing comprises an inlet portion and an outlet portion.

5. The filter assembly of claim 4, wherein the inlet portion is circular, and wherein the outlet portion is circular.

6. The filter assembly of claim 5, wherein the inlet portion is axially aligned with the outlet portion.

7. The filter assembly of claim 6, wherein the filter housing comprises a connecting portion between the inlet portion and the outlet portion.

8. The filter assembly of claim 5, wherein the connecting portion is at least partially tapered between the inlet portion and the outlet portion.

9. The filter assembly of claim 4, wherein the inlet portion comprises a larger major dimension than the outlet portion.

10. The filter assembly of claim 1, wherein the filter assembly is configured to be coupled to an evacuation component, wherein the evacuation component comprises a dental high vacuum evacuation (HVE) line.

11. The filter assembly of claim 1, wherein the inlet portion comprises a lip that forms an opening in the inlet portion.

12. The filter assembly of claim 1, wherein at least 75% of an outer surface of the filter medium is exposed to the external environment.

13. The filter assembly of claim 1, wherein the filter assembly is configured to remove aerosols from the external environment.

14. The filter assembly of claim 13, wherein the filter housing comprises a flow passage connecting the inlet portion and the outlet portion to facilitate removal of the aerosols from the external environment.

15. The filter assembly of claim 14, wherein the aerosols removed from the external environment pass through the filter medium, through the flow passage, and into an evacuation component coupled to the filter assembly.

16. The filter assembly of claim 1, wherein the filter medium comprises an antibacterial agent, an antimicrobial agent, an antiviral agent, or a combination thereof.

17. The filter assembly of claim 1, wherein the filter medium prevents backflow of bacteria, viruses, other contaminants or chemicals (e.g., blood, mercury, etc.) through the filter medium into the external environment.

18. The filter assembly of claim 1, wherein the filter assembly is configured to remove aerosols from the external environment when the filter assembly is placed at a distance of between 0 mm and 350 mm from the surgical location of the medical procedure.

19. The filter assembly of claim 18, wherein the surgical location is the mouth of a subject for a dental operation.

20. The filter assembly of claim 19, wherein the surgical location is an incision on a subject for a medical operation.
